SEQUENCE LISTING (4 sequences)

SEQ ID NO: 1 - DNA, 240 bp, Homo Sapiens
ttacagtgctcccacacaactgaatcccgaactgccttggcaaagcaactgcaaatgtgctatatataaaggagtccatgatgtagtgtttaatgcagaagcaatttatagaaggggttgtgtaatattacctcatttttattttaaaaagtatccacacatcaaacactatatttctttgtacttaaaaatatttttaaaataaaacataatggtgatgaatgggtggatgagtggaaa

SEQ ID NO: 2 - DNA, 131 bp, Homo Sapiens
taggatgtcaggcagatggaatattttcatctctttgtagagagaagcattggtgattgctcctgttccaaacaaaagtggctcaaggaagaaccagtgacagtctctgaaaactcaaaactccacaggag

SEQ ID NO: 3 - DNA, 151 bp, Homo Sapiens
ctatagtagacaacatacgaccacgctaaacccaaatacatctcccaggattgtaagtaggggtcaagaaaatagtgatcatttgtgccataaaagtgctcaggtcccacagcaacaatatcattcaaactgcaattaaaacatacacaca

SEQ ID NO: 4 - DNA, 150 bp, Homo Sapiens
gttttaatccagagaaaatatgagagaagaaaagctgcttggcttggatcaagagaagtcttcctcctcttctgccctgagtgtggtcctgaccctgtggctttagtcataccccacaaagatctggggttagtctaagcactgtgcatc

(19) United States
(12) Patent Application Publication
(10) Pub. No.: US 2009/0029367 A1
(43) Pub. Date: Jan. 29, 2009

(54) POLYMORPHISMS IN PON1 ARE ASSOCIATED WITH ELEVATED ALANINE AMINOTRANSFERASE LEVELS AFTER XIMELAGATRAN OR TACRINE ADMINISTRATION

(75) Inventors: Olof Bengtsson, Molndal (SE); Ellen Brown, Cheshire (GB); Stefan Carlsson, Molndal (SE); Neil James Gibson, Cheshire (GB); Ansar Jawaid, Cheshire (GB); Andreas Kindmark, Molndal (SE); Ruth Eleanor March, Cheshire (GB)

Correspondence Address: ASTRAZENECA R&D BOSTON, 35 GATEHOUSE DRIVE, WALTHAM, MA, 02451-1215, US

(73) Assignee: AstraZeneca AB, Sodertälje (SE)

(21) Appl. No.: 12/067,864

(22) PCT Filed: Oct. 3, 2006
(86) PCT No.: PCT/GB2006/003662

§ 371 Date: Mar. 24, 2008

(30) Foreign Application Priority Data
Oct. 5, 2005 (GB) 0520234.6

(51) Int. Cl. C12Q 1/68

(52) U.S. Cl. 435/6

(57) ABSTRACT

This invention relates to a method for administering a pharmaceutically useful anticoagulant drug to certain suitable patients and a method for identifying those patients suitable for receiving the drug. In particular, the invention surrounds the identification of an association between certain SNPs in the PON1 gene and susceptibility to increased levels of alanine aminotransferase (ALAT) following ximelagatran or tacrine administration. Thus, this invention relates to methods for predicting susceptibility to elevated ALAT following ximelagatran or tacrine administration and to methods for administering a pharmaceutically useful anticoagulant drug to certain suitable patients.

### FIELD OF THE INVENTION

The present invention is based on the discovery of a genetic association between certain polymorphisms in paraoxonase-1 (PON-1), an arylesterase with multiple biological activities, and incidence of elevated ALAT following therapeutic drug administration. The inventors have found that certain single nucleotide polymorphisms are predictive of an increased likelihood of elevated ALAT following administration of therapeutic drugs likely to interact with paraoxonase, such as those involved in the modulation of lipid or cholinesterase pathways. Thus, in particular, this invention relates to a method for administering pharmaceutically useful anticoagulant or anticholinesterase drugs to certain suitable patients and a method for identifying those patients suitable for receiving the drug.

### BACKGROUND

Blood coagulation is the key process involved in both haemostasis (i.e. the prevention of blood loss from a damaged vessel) and thrombosis (i.e. the formation of a blood clot in a blood vessel, sometimes leading to vessel obstruction).

Coagulation is the result of a complex series of enzymatic reactions. One of the ultimate steps in this series of reactions is the conversion of the proenzyme prothrombin to the active enzyme thrombin.

Thrombin is known to play a central role in coagulation. It activates platelets, leading to platelet aggregation, converts fibrinogen into fibrin monomers, which polymerise spontaneously into fibrin polymers, and activates factor XIII, which in turn crosslinks the polymers to form insoluble fibrin. Furthermore, thrombin activates factor V and factor VIII leading to a “positive feedback” generation of thrombin from prothrombin.

By inhibiting the aggregation of platelets and the formation and cross-linking of fibrin, effective inhibitors of thrombin would therefore be expected to exhibit antithrombotic activity. In addition, antithrombotic activity would be expected to be enhanced by effective inhibition of the positive feedback mechanism.

The development of low molecular weight inhibitors of thrombin has been described by Claesson (Blood Coagul. Fibrin. 5:411, 1994), and certain thrombin inhibitors based on peptide derivatives have been disclosed, for example, in European Patent Application 0 669 317 and International Patent Applications WO 95/23609, WO 95/35309, WO 96/25426 and WO 94/29336.

The latter application discloses the peptide derivatives R^{a}OOC—CH₂—(R)Cgl-Aze-Pab-H, wherein R^{a} represents H, benzyl or C₁₋₆ alkyl. When R^{a} represents H the compound is known as melagatran.

The compound known as ximelagatran (EtOOC—CH₂—(R)Cgl-Aze-Pab-OH) has been developed for use, for example, in orthopaedic surgery and in atrial fibrillation. Upon oral administration, ximelagatran is metabolised to the active thrombin inhibitor melagatran. Further details on ximelagatran and its preparation are contained in, for example, WO 97/23499.

For reference, Aze=S-Azetidine-2-carboxylic acid; Cgl=cyclohexylglycine; H-Pab-H=1-amidino-4-aminomethyl benzene; Pab-OH=4-aminomethyl-benzamidoxime (4-aminomethyl-1-(amino-hydroxyiminomethyl)benzene).

Phase III clinical trials have been performed using fixed doses of melagatran and ximelagatran for the prevention of VTE in hip or knee replacement surgery. In addition, clinical trials have been performed using ximelagatran for the treatment and long-term secondary prevention of VTE, and for the prevention of stroke in patients with non-valvular atrial fibrillation. Ximelagatran has also been tested for secondary thrombosis prophylaxis post-myocardial infarction/acute coronary syndrome (ACS).

Alanine aminotransferrase (ALAT) is an enzyme mostly expressed in the liver (EC 2.6.1.2). It is also called serum glutamate pyruvate transaminase (SGPT) or alanine transaminase (ALT). This enzyme is release into the plasma by liver cell death, which is a normal event. However, when liver cell death increases, ALAT levels rise above the normal range. The spill-over of this enzyme into blood is routinely measured as a marker of abnormal liver-cell damage. For example, alcoholic or viral hepatitis will increase ALAT levels, as will severe congestive heart failure. ALAT is also markedly raised in hepatitis and other acute liver damage. An elevated ALAT in the presence of normal levels of plasma alkaline phosphatase helps distinguish liver disease caused by liver-cell damage from diseases caused by problems in biliary ducts. Elevations of ALAT are normally measured in multiples of the upper limit of normal (ULN), with a reference range of 15-45 U/L in most laboratories. In 1987, in a study of 19,877 healthy Air Force recruits, only 99 (0.5%) had confirmed ALAT elevations (as reviewed in Green & Flamm (2002) Gastroenterology 123:1367-1384).

During longer-term treatment with ximelagatran (>35 days) 7.9% of patients exhibited levels of alanine aminotransferase (ALAT) 3-fold or more above the upper limit of normal (≧3×ULN) compared with 1.2% in the comparator groups. The increase in ALAT values with ximelagatran usually occurred within the first 6 months of treatment and were mainly asymptomatic. Furthermore, these increases in ALAT were reversible in most patients regardless of whether treatment was continued or discontinued. Subject to the future regulatory approval of ximelagatran, regular liver function testing (LFT) using an appropriate algorithm may be required if ximelagatran is used for treatment periods exceeding a month. Studies are currently ongoing to try and establish the mechanism of the ALAT elevations, and their hepatic and overall clinical significance.

Tacrine hydrochloride is a reversible cholinesterase inhibitor, known chemically as 1,2,3,4-tetrahydro-9-acridinamine monohydrochloride monohydrate. Tacrine hydrochloride is commonly referred to in the clinical and pharmacological literature as THA. It has an empirical formula of C₁₃H₁₄N₂.HCl.H₂O and a molecular weight of 252.74. Cholinesterase inhibitors inhibit the action of acetylcholinesterase, the enzyme responsible for the destruction of acetylcholine. Acetylcholine is one of several neurotransmitters in the brain, chemicals that nerve cells use to communicate with one another. Reduced levels of acetylcholine in the brain are believed to be responsible for some of the symptoms of Alzheimer's disease. By blocking the enzyme that destroys acetylcholine, rivastigmine increases the concentration of acetylcholine in the brain, and this increase is believed to be responsible for the improvement in thinking seen with tacrine. The most common side effect of tacrine is an increase in alanine aminotransferase (ALAT) as a result of liver damage. Hence, patients treated with tacrine are tested for ALAT on a weekly basis. If there is an increase in blood ALAT, the dosage of tacrine can be reduced.

Accordingly, it is desirable to identify which patients are likely to experience raised ALAT levels when receiving therapeutic drugs that are likely to interact with paraoxonase, such as ximelagatran or tacrine. The sub-groups of individuals identified as having increased or decreased likelihood of experiencing elevated ALAT following ximelagatran or tacrine administration, can be used, inter alia, for targeted clinical trial programs and possibly also pharmacogenetic therapies.

This invention results from the discovery that members of a sub-population of patients on ximelagatran or tacrine therapy that experience substantial (≧3-fold) elevated alanine aminotransferase (ALAT) liver enzyme levels have particular genetic profiles. In particular, the inventors have identified a genetic association between elevated ALAT following administration of drugs likely to interact with paraoxonase (such as ximelagatran or tacrine) and particular SNPs in the paroxonase (PON1) gene.

Paroxonase (paraoxonase; PON1; EC 3.1.1.2) is an arylesterase that is capable of hydrolyzing paroxon to produce p-nitrophenol. Paroxon is an organophosphorus anticholinesterase compound, used topically in the treatment of glaucoma. It is produced in vivo in mammals by microsomal oxidation of the insecticide parathion. Parathion is inert until transformed to paroxon.

Paraoxonase-1 (PON-1) has multiple biological activities. It is a potent endogenous cholinesterase inhibitor. By hydrolyzing paraoxon and other organophosphates, PON-1 provides protection against exogenous organophosphate poisoning. In addition, PON-1 is also largely responsible for the antioxidant activity of high-density lipoproteins. Experiments with mice lacking the apoE gene have shown that exogenous PON-1 is able to reverse the oxidative stress in macrophages, suggesting that PON-1 might also have potentially important anti-inflammatory activities.

Drugs that interact with PON1 may be deduced from the structural requirements for PON1's lactonase activity, such as lactone- and carbonate ester-containing drugs or prodrugs. For example, it has been found that the diuretic spironolactone and some hydroxymethylglutaryl-CoA reductase inhibitors (mevastatin, lovastatin, and sinivastatin) are hydrolyzed by PON1. Other drugs that interact with PON1 include the cholinesterase inhibitors used for treatment of Alzheimer's disease, such as tacrine, donepezil, and rivastigmine. Examples of drugs known to interact with paraoxonase are shown in Table 1.

TABLE 1
Examples of drugs known to interact with paraoxonase

| Drug name | Pathway | Reference |
|---|---|---|
| Ximelagatran | Lipid | In-house data (Example 1) |
| Tacrine | Cholinesterase | In-house data (Example 2) |
| Donepezil | Cholinesterase | 1 |
| Rivastigmine | Cholinesterase | 1 |
| Prulifloxacin | Anti-bacterial | 2 |
| Spironolactone | Aldosterone receptor | 2 |
| | antagonists | |
| Lovastatin, | Lipid | 2 |
| Simvastatin | Lipid | 2 |
| Mevastatin | Lipid | 3 |
| Glucocorticoid | Pulmonary | 3 |
| lactone antedrugs | | |

### References

1. Pola R, Flex A, Ciaburri M, Rovella E, Valiani A, Reali G, Silveri M C, Bernabei R. Neurosci Lett. 2005 Jul. 15; 382(3):338-41.
2. Draganov D I and La Du B N. J Naunyn-Schmiedeberg's Arch Pharmacol 2004 369: 78˜88.
3. Billecke S, Draganov D, Counsell R, Stetson P, Watson C, Hsu C, La Du B N. Drug Metab Dispos. 2000 November; 28(11):1335-42.

The complete coding sequence of PON1 (AF539592) was first sequenced from chromosome 7 and submitted to the EMBL/GenBank/DDBJ databases by Rieder et al. (2002).

PON1 is known to contain SNPs in the 5′ region that cause changes in transcription levels (Brophy et al. Am J Hum Genet 68, 1428-1436 (2001)). In addition, a SNP in exon 6 (rs662) that alters the amino acid at position 192 of the enzyme (Q192R) affects the activity of the enzyme against various substrates (O'Leary et al, Pharmacogenet Genomics 15: 51-60 (2005)). The C allele at position 52 of SEQ ID NO:3 encodes the R form of the enzyme, which is associated with reduced activity. A haplotype conferring increased transcription of an allele with reduced enzymatic activity could have profound effects on its interaction with therapeutic agents.

The PON1 SNPs showing association according to the present invention include (in order): afd4084667/rs 2299257, afd4084666/rs 1157745, afd0513208/rs 662 and afd0513205/rs 2269829. Each of these SNPs is in strong linkage disequilibrium with other members of the group.

The identification of genetic markers that are closely associated with a predisposition to develop particular pharmacological effects, such as elevated alanine aminotransferase (ALAT) liver enzyme levels, can be used to design diagnostic or prognostic genetic tests.

The invention also relates to methods and materials for analysing allelic variation in the PON1 gene, and to the use of PON1 polymorphisms in the identification of an individual's likelihood to experience certain pharmacological effects when being treated with a drug likely to interact with paraoxonase (such as ximelagatran or tacrine).

The invention also relates to methods and materials for stratifying patients to be treated with ximelagatran or tacrine into those that are likely or unlikely of experiencing elevated ALAT levels following ximelagatran or tacrine treatment, thus offering the ability to make informed decisions about whether or not a particular patient or sub-patient population should be treated with the drug.

The sub-groups of individuals identified as having increased or decreased likelihood of experiencing elevated ALAT following ximelagatran or tacrine administration, can be used, inter alia, for targeted clinical trial programs and possibly also pharmacogenetic therapies.

By elevated ALAT we mean, for example ≧3-fold upper limit of normal (as reviewed in Green & Flamm, ibid).

The location of the polymorphisms can be precisely mapped by reference to published EMBL (or other sequence database) sequence accession numbers (i.e. see above), alternatively, the person skilled in the art can precisely identify the location of the polymorphism in the particular gene simply by provision of flanking sequence adjacent the polymorphism sufficient to unambiguously locate the polymorphism. Provision of 10 or more nucleotides each side of the polymorphism should be sufficient to achieve precise location mapping of the particular polymorphism.

The use of knowledge of polymorphisms to help identify patients most suited to therapy with particular pharmaceutical agents is often termed “pharmacogenetics”. Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43:254; Marshall (1997), Nature Biotechnology. 15:1249; International Patent Application WO 97/40462, Spectra Biomedical; and Schafer et al, (1998), Nature Biotechnology. 16:33.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas. The presence of a particular base at a polymorphism position will be represented by the base following the polymorphism position. For (a hypothetical) example, the presence of adenine at position 300 will be represented as: 300A.

### DISCLOSURE OF THE INVENTION

The invention is based on the finding of an association between individuals that possess an adenine base (A) at polymorphism site rs2299257 (position 102 according to SEQ ID NO: 1) and normal ALAT enzyme levels. Whereas, those that do not possess a copy of this polymorphic allele are more likely to experience >3-fold elevated ALAT levels in blood plasma.

Thus, according to a first aspect of the present invention, there is provided a method of diagnosis comprising:
a) providing a biological sample from a human identified as being in need of treatment with a drug likely to interact with paraoxonase, wherein the sample comprises a nucleic acid encoding PON1 gene;
b) testing the nucleic acid for the presence, on at least one allele, of either
i) a nucleotide A at the position corresponding to position 102 of SEQ ID NO: 1, or
ii) an allele of a polymorphism in linkage disequilibrium with a D′>0.9 with (i); and
c) if either (i) or (ii) is found in at least one allele, diagnosing the human as being in the low likelihood category of having raised liver enzymes after treatment with the drug. In a particular embodiment the drug likely to interact with paraoxonase is ximelagatran or tacrine.

A drug likely to interact with paraoxonase includes a drug that is known to interact with paraoxonase, and includes those in Table 1.

In particular embodiments, the allele of a polymorphism in linkage disequilibrium with a D′>0.9 with the A polymorphism at position 102 of SEQ ID NO: 1 is selected from the group consisting of: G at position 52 of SEQ ID NO:2, T at position 52 of SEQ ID NO:3, and A at position 51 of SEQ ID NO:4. These represent alleles of polymorphisms rs1157745, rs662 and rs2269829, which are in significant linkage disequilibrium with position 102 of SEQ ID NO:1 (D′=1 for all polymorphisms, see Table 2).

Thus, individuals that possess one or more of: A at position 102 of SEQ ID NO: 1, G at position 52 of SEQ ID NO:2, T at position 52 of SEQ ID NO:3, and A at position 51 of SEQ ID NO:4, on at least one chromosomal copy are less likely to experience raised liver enzymes, in particular greater than a 3-fold elevated ALAT level following administration of a drug likely to interact with paraoxonase (such asximelagatran or tacrine), relative to the level before administration, and are therefore in the “low likelihood” category.

According to a further aspect of the invention there is provided a method of genotyping an individual in order to determine the individual's potential likelihood to experience elevated ALAT following administration with a drug likely to interact with paraoxonase, comprising determining the nucleotide present at a polymorphic position selected from the group consisting of: position 102 of SEQ ID NO: 1, or an allele of a polymorphism in linkage disequilibrium with D′>0.90 thereto, on one or both chromosomal copies, in a sample that has previously been removed from the individual, and determining the individual's likelihood of experiencing elevated ALAT following drug administration according to the nucleotide present. In a particular embodiment the drug likely to interact with paraoxonase is ximelagatran or tacrine.

According to another aspect of the invention there is provided a method for screening an individual for a genetic predisposition to elevated ALAT following ximelagatran or tacrine administration, comprising analysing the individual's nucleic acid in a sample removed from the individual for the presence or absence of an adenine (A) at position 102 according to SEQ ID NO: 1, or an allele of a polymorphism in linkage disequilibrium with D′>0.90 thereto, and determining the status of the individual by reference to the particular base present.

As noted above, SNPs in linkage disequilibrium with rs 2299257 include: rs1157745, rs662 and rs2269829. The alleles that associate with reduced likelihood to experience elevated ALAT levels following ximelagatran or tacrine administration (i.e. low likelihood category) include A at rs 2299257, G at rs1157745, T at rs662 and A at rs2269829.

Alleles that associate with elevated ALAT include: C at rs 2299257, T at rs1157745, C at rs662 and G at rs2269829. Thus, the status of the individual, in terms of likelihood of experiencing elevated ALAT following ximelagatran or tacrine administration can be determined according to presence or absence of the particular alleles identified above and whether or not they are present in one or two copies.

Single nucleotide polymorphisms (SNPs) represent one of the most common forms of genetic variation. These polymorphisms appear when a single nucleotide in the genome is altered (such as via substitution, addition or deletion). For example, if at a particular chromosomal location one member of a population has an adenine and another member has a thymine at the same position, then this position is a single nucleotide polymorphic site. Each version of the sequence with respect to the polymorphic site is referred to as an “allele” of the polymorphic site. SNPs tend to be evolutionarily stable from generation to generation and, as such, can be used to study specific genetic abnormalities throughout a population. If SNPs occur in the protein-coding region it can lead to the expression of a variant, sometimes defective, form of the protein that may lead to development of a genetic disease. Such SNPs can therefore serve as effective indicators of the genetic disease. Some SNPs may occur in non-coding regions, but nevertheless, may result in differential or defective splicing, or altered protein expression levels. SNPs can therefore be used as diagnostic tools for identifying individuals with a predisposition for certain diseases, genotyping the individual suffering from the disease in terms of the genetic causes underlying the condition, and facilitating drug development based on the insight revealed regarding the role of target proteins in the pathogenesis process. Clinical trials have shown that patient response to treatment with pharmaceuticals, in terms of efficacy and safety (side effects etc.) is often heterogeneous. It is thus well known that SNPs can also be used as diagnostic or prognostic tools for gauging drug efficacy or safety.

A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs.

The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine. Ann Hum Genet (1998) 62:481-90, De Stefano V, Dekou V, Nicaud V, Chasse J F, London J, Stansbie D, Humphries S E, and Gudnason V; and Variation at the von willebrand factor (vWF) gene locus is associated with plasma vWF:Ag levels: identification of three novel single nucleotide polymorphisms in the vWF gene promoter. Blood (1999) 93:4277-83, Keightley A M, Lam Y M, Brady J N, Cameron C L, Lillicrap D).

As noted above, the rs662 SNP alters the amino acid at position 192 of the enzyme (Q192R) and affects activity of the enzyme against various substrates (O'Leary et al. supra). Accordingly, detection of the presence of this SNP can also be performed by detection of the altered amino acid (i.e. presence of arginine at position 192) or by enzymatic activity assays. Thus, detection of the C nucleotide at position 52 (according to SEQ ID NO: 3), and its use in the methods and kits of the invention, can be undertaken indirectly via enzymatic activity determination and or the presence of the arginine substitution at amino acid position 192 (e.g. see Example 3).

According to another aspect of the invention there is provided a method for subtyping human individuals according to their likelihood status of experiencing elevated ALAT following administration of a drug likely to interact with paraoxonase, comprising the steps of:
a) treating nucleic acid from a sample that has been removed from the individual so as to identify the nucleotides present at one or more of the PON1 gene SNPs selected from the group consisting of rs2299257, rs1157745, rs662 and rs2269829; and
b) assigning the individual to a particular subtype based on likelihood of experiencing elevated ALAT following drug administration, according to the nucleotide(s) detected in step a).

In a particular embodiment the drug likely to interact with paraoxonase is ximelagatran or tacrine.

The test sample (the nucleic acid containing sample) is conveniently a sample of blood, plasma, bronchoalveolar lavage fluid, saliva, sputum, cheek-swab or other body fluid or tissue (such as a biopsy sample) obtained from an individual that contains nucleic acid molecules. The nucleic acid containing sample that is to be analysed can either be a treated or untreated biological sample isolated from the individual. A treated sample, may be for example, one in which the nucleic acid contained in the original biological sample has been isolated or purified from other components in the sample (tissues, cells, proteins etc), or one where the nucleic acid in the original sample has first been amplified, for example by polymerase chain reaction. Thus, it will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

For the avoidance of doubt, the methods of the invention do not involve diagnosis practised on the human body. The methods of the invention are preferably conducted on a sample that has previously been removed from the individual. The kits of the invention, however, may include means for extracting the sample from the individual. When specifying a particular nucleotide at an allele position it is important to appreciate which of the two complementary strands of nucleic acid the nucleotide resides on. For example, a G on the positive strand will correspond to a C on the negative (reverse) strand. The correct strand may also be deduced by the nucleotide sequence adjacent the allele, by reference to the sequence listings provided herein.

The ability to identify patients that have increased likelihood of experiencing elevated ALAT following ximelagatran or tacrine treatment allows the patient or their physician to assess their suitability for treatment with ximelagatran or tacrine. It also allows, for example, the option to include or exclude such individuals in clinical studies.

The presence of specific “elevated ALAT susceptibility markers” however does not mean that the individual will definitely experience elevated ALAT following ximelagatran or tacrine administration. It merely suggests that the individual compared to the population as a whole has a higher likelihood of experiencing elevated ALAT following ximelagatran or tacrine administration.

According to a further aspect of the invention there is provided a diagnostic or prognostic method of predicting susceptibility to produce elevated (≧3-fold) ALAT following ximelagatran or tacrine administration, based on the detection of the particular nucleotide present at an “elevated ALAT susceptibility marker” selected from the group consisting of: rs2299257, rs1157745, rs662 and rs2269829, in an individual.

According to a further aspect of the invention there is provided a method of diagnosing or predicting susceptibility to elevated (≧3-fold) ALAT following ximelagatran or tacrine administration, in an individual, comprising determining the presence or absence in a sample from said individual of an “elevated ALAT susceptibility marker” selected from the group consisting of: an cytosine at allele rs2299257 (position 102 according to SEQ ID NO: 1), a thymine at allele rs1157745 (position 52 according to SEQ ID NO: 2), a cytosine at allele rs662 (position 52 according to SEQ ID NO: 3) and an guanine at allele rs2269829 (position 51 according to SEQ ID NO: 4), wherein the presence of said elevated ALAT susceptibility marker is diagnostic or predictive of susceptibility to experience elevated (≧3-fold) ALAT following ximelagatran or tacrine administration to said individual.

The inventors have identified that each of 4 specific SNPs within the PON1 gene are associated with elevated ALAT blood levels following ximelagatran or tacrine administration. Each of these alleles is in strong linkage disequilibrium with the other alleles of the group (D′ of 1.0).

Thus, according to another aspect of the invention there is provided a method of diagnosing or predicting an individual's susceptibility to elevated ALAT following ximelagatran or tacrine administration to said individual, comprising determining the presence or absence in a sample removed from said individual of a cytosine (C) nucleotide at allele rs2299257 (position 102 according to SEQ ID NO: 1), or an allele in linkage disequilibrium with D′>0.9 therewith, wherein the presence of said nucleotide is diagnostic or predictive of susceptibility to elevated ALAT following ximelagatran or tacrine administration.

The SNPs of the invention demonstrate significant association to experiencing elevated ALAT following ximelagatran or tacrine administration. However, the person skilled in the art will appreciate that a diagnostic test consisting solely of a SNP of the invention will not be diagnostic of raised ALAT for any particular individual following ximelagatran or tacrine administration. Nevertheless, in line with future developments we envisage that the SNPs of the present invention could form part of a panel of markers that in combination will be predictive of elevated ALAT following ximelagatran or tacrine administration for any individual, within normal clinical standards sufficient to influence clinical practice.

It will be apparent to the person skilled in the art that the various aspects of the invention that relate to or refer to ximelagatran or tacrine are equally applicable to other drugs that are likely to interact with paraoxonase.

Because there are two copies of each chromosome (a maternal and paternal copy), at each chromosomal location the human may be homozygous for an allele or the human may be a heterozygote. If the individual is heterozygous the presence of both alternate alleles will be present.

It will be apparent to the person skilled in the art that there are a large number of analytical procedures, which may be used to detect the presence or absence of variant nucleotides at one or more polymorphic positions of the invention. In general, the detection of allelic variation requires a mutation discrimination technique, optionally an amplification reaction and optionally a signal generation system. List 1 lists a number of mutation detection techniques, some based on the PCR. These may be used in combination with a number of signal generation systems, a selection of which are listed in List 2. Further amplification techniques are listed in List 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

### Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| Bp | base pair |
| CMC | Chemical mismatch cleavage |
| COPS | Competitive oligonucleotide priming system |
| DGGE | Denaturing gradient gel electrophoresis |
| ELISA | Enzyme Linked ImmunoSorbent Assay |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

### List 1—Mutation Detection Techniques

General: DNA sequencing, Sequencing by hybridisation
Scanning: PTT, SSCP, DGGE, TGGE, Cleavase, Heteroduplex analysis, CMC, Enzymatic mismatch cleavage

### Hybridisation Based

Solid phase hybridisation: Dot blots, MASDA, Reverse dot blots, Oligonucleotide arrays (DNA Chips).

Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)

Extension Based: ARMS™—allele specific amplification, ALEX™—European Patent No. EP 332435 B1 (Zeneca Limited), COPS—Gibbs et al (1989), Nucleic Acids Research, 17, 2347.

### Incorporation Based: Mini-sequencing, APEX

Restriction Enzyme Based: RFLP, Restriction site generating PCR

### Ligation Based: OLA

Other: Invader assay

### List 2—Signal Generation or Detection Systems

Fluorescence: FRET, Fluorescence quenching, Fluorescence polarisation—United Kingdom Patent No. 2228998 (Zeneca Limited)
Other: Chemiluminescence, Electrochemiluminescence, Raman, Radioactivity, Colorimetric, Hybridisation protection assay, Mass spectrometry

### List 3—Further Amplification Methods

SSR, NASBA, LCR, SDA, b-DNA

### List 4—Protein Variation Detection Methods

### Immunoassay

### Immunohistology

Peptide sequencing

Thus, the presence or absence of a ximelagatran or tacrine induced raised ALAT predisposing SNP useful in the invention can be determined, for example, using enzymatic amplification of nucleic acid from the individual. In one embodiment, the presence or absence of a particular raised ALAT predisposing SNP allele is determined using polymerase chain reaction (PCR). In a further embodiment the PCR is performed with allele-specific oligonucleotide primers capable of discriminating between the different bases at a particular allele, such as using amplification refractory mutation system (ARMS™—allele specific amplification). In a further embodiment, the PCR is performed using one or more fluorescently labelled probes or using one or more probes which include a DNA minor groove binder. The presence or absence of a particular SNP allele can also be determined, for example, by sequence analysis.

The nucleic acid sequence method for diagnosis is preferably one which is determined by a method selected from amplification refractory mutation system, restriction fragment length polymorphism and primer extension. In another embodiment, the nucleotide present at each polymorphic position is determined by sequence analysis, such as by dideoxy sequencing.

Preferred mutation detection techniques include ARMS™—allele specific amplification, ALEX™, COPS, Taqman, Molecular Beacons, RFLP, and restriction site based PCR and FRET techniques. Immunoassay techniques are known in the art e.g. A Practical Guide to ELISA by D M Kemeny, Pergamon Press 1991; Principles and Practice of Immunoassay, 2^{nd} edition, C P Price & D J Newman, 1997, published by Stockton Press in USA & Canada and by Macmillan Reference in the United Kingdom.

Particularly preferred methods include ARMS™—allele specific amplification, OLA and RFLP based methods. The allele specific amplification technique known in the art as ARMS™—allele specific amplification is an especially preferred method.

ARMS™—allele specific amplification (described in European patent No. EP-B-332435, U.S. Pat. No. 5,595,890 and Newton et al. (Nucleic Acids Research, Vol. 17, p. 2503; 1989)), relies on the complementarity of the 3′ terminal nucleotide of the primer and its template. The 3′ terminal nucleotide of the primer being either complementary or non-complementary to the specific mutation, allele or polymorphism to be detected. There is a selective advantage for primer extension from the primer whose 3′ terminal nucleotide complements the base mutation, allele or polymorphism. Those primers which have a 3′ terminal mismatch with the template sequence severely inhibit or prevent enzymatic primer extension. Polymerase chain reaction or unidirectional primer extension reactions therefore result in product amplification when the 3′ terminal nucleotide of the primer complements that of the template, but not, or at least not efficiently, when the 3′ terminal nucleotide does not complement that of the template.

In a further aspect, the detection/diagnostic methods of the invention, are used to assess the predisposition and/or susceptibility of an individual to experience elevated ALAT following ximelagatran or tacrine administration.

In a further diagnostic aspect of the invention the presence or absence of variant nucleotides is detected by reference to the loss or gain of, optionally engineered, sites recognised by restriction enzymes. The person of ordinary skill will be able to design and implement diagnostic procedures based on the detection of restriction fragment length polymorphism due to the loss or gain of one or more of the restriction sites due to the presence of a polymorphism.

According to a further aspect of the invention there is provided the use of an “elevated ALAT susceptibility marker” selected from the group consisting of markers: rs2299257, rs1157745, rs662 and rs2269829, as a tool for the prediction of elevated ALAT following ximelagatran or tacrine administration to an individual.

The invention further provides nucleotide primers which detect the PON1 gene polymorphisms of the invention. Such primers can be of any length, for example between 8 and 100 nucleotides in length, but will preferably be between 12 and 50 nucleotides in length, more preferable between 17 and 30 nucleotides in length. Preferably, such primers are allele specific primers capable of detecting one of the associated PON1 gene polymorphisms identified herein.

An allele specific primer is used, generally together with a constant primer, in an amplification reaction such as a PCR reaction, which provides the discrimination between alleles through selective amplification of one allele at a particular sequence position e.g. as used for ARMS™—allele specific amplification assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer. Often the nucleotide at the −2 and/or −3 position (relative to the 3′ terminus) is mismatched in order to optimise differential primer binding and preferential extension from the correct allele discriminatory primer only.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting one of the associated PON1 gene polymorphism of the invention.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection, such as in Molecular Beacons. Single stranded oligonucleotides corresponding to SEQ ID NOs: 1-4 or their complement, could be used as probes to detect the particular polymorphism at the central position. The probe would bind more efficiently to a target sequence that possessed the particular complementary polymorphism base at this central (polymorphism) location than one with a base mismatch.

According to another aspect of the present invention there is provided an allele specific primer or an allele specific oligonucleotide probe capable of detecting a PON1 gene polymorphism at one of the positions defined herein.

According to another aspect of the invention there is provided a kit for screening for a genetic predisposition to elevated ALAT levels following ximelagatran or tacrine administration, which kit comprises:
(i) reagents for analysing one or more of the PON1 gene SNPs rs2299257, rs1157745, rs662 and rs2269829, and optionally,
(ii) means for collecting a nucleic acid sample or nucleic acid containing sample.

According to another aspect of the invention there is provided an in vitro diagnostic kit for determining the identity of one or more of SNPs rs2299257, rs1157745, rs662 and rs2269829, in the human PON1 gene, said kit comprising components for the determination of the nucleotide present at said SNP locations.

In particular embodiments of the invention, the kit components for determining said SNPs include allele-specific amplification primers or allele-specific hybridisation probes capable of determining the identity of the nucleotide bases at the SNP locations.

According to another aspect of the invention there is provided a kit comprising one or more diagnostic primer(s) and/or one or more allele-specific oligonucleotide probes(s) capable of determining the identity of the nucleotide present at one or more of the following SNPs: rs2299257, rs1157745, rs662 and rs2269829, in the human PON1 gene.

The diagnostic kits may comprise appropriate packaging and instructions for use in the methods of the invention. Such kits may further comprise appropriate buffer(s) and polymerase(s) such as thermostable polymerases, for example taq polymerase. Such kits may also comprise companion primers and/or control primers or probes. A companion primer is one that is part of the pair of primers used to perform PCR. Such primer usually complements the template strand precisely.

The SNPs of the invention represent a valuable information source with which to characterise individuals in terms of, for example, their identity and susceptibility to side effects following treatment with particular drugs. These SNPs, including nucleotide sequences related to these, may be stored in a computer readable medium. The polymorphism referred to herein are particularly useful as components in databases useful for sequence identity, genome mapping, pharmacogenetics and other search analyses. Generally, the sequence information relating to the nucleic acid sequences and polymorphisms of the invention may be reduced to, converted into or stored in a tangible medium, such as a computer disk, preferably in a computer readable form. For example, chromatographic scan data or peak data, photographic scan or peak data, mass spectrographic data, sequence gel (or other) data.

The computer readable medium may be used, for example, in homology searching, mapping, haplotyping, genotyping or pharmacogenetic analysis. The computer readable medium can be any composition of matter used to store information or data, including, for example, floppy disks, tapes, chips, compact disks, digital disks, video disks, punch cards and hard drives.

The compounds of WO 94/29336 and the prodrug compounds of WO 97/23499 are expected to be useful in those conditions where inhibition of thrombin is required.

In particular, the compounds of WO 97/23499, and ximelagatran in particular, are thus indicated both in the therapeutic and/or prophylactic treatment of thrombosis and hypercoagulability in blood and tissues of animals including man.

It is known that hypercoagulability may lead to thrombo-embolic diseases. Thrombo-embolic diseases which may be mentioned include: activated protein C resistance, such as the factor V-mutation (factor V Leiden), and inherited or acquired deficiencies in antithrombin III, protein C, protein S, heparin cofactor II. Other conditions known to be associated with hypercoagulability and thrombo-embolic disease include circulating antiphospholipid antibodies (Lupus anticoagulant), homocysteinemi, heparin induced thrombocytopenia and defects in fibrinolysis. The compounds of WO 97/23499, and ximelagatran in particular, are thus indicated both in the therapeutic and/or prophylactic treatment of these conditions.

The compounds of WO 97/23499, and ximelagatran in particular, are further indicated in the treatment of conditions where there is an undesirable excess of thrombin without signs of hypercoagulability, for example in neurodegenerative diseases such as Alzheimer's disease.

Particular disease states which may be mentioned include: the therapeutic and/or prophylactic treatment of venous thrombosis and pulmonary embolism, arterial thrombosis (eg in myocardial infarction, unstable angina, thrombosis-based stroke and peripheral arterial thrombosis) and systemic embolism usually from the atrium during arterial fibrillation or from the left ventricle after transmural myocardial infarction.

Moreover, the compounds of WO 97/23499, and ximelagatran in particular, are expected to have utility in prophylaxis of re-occlusion (i.e. thrombosis) after thrombolysis, percutaneous trans-luminal angioplasty (PTA) and coronary bypass operations; the prevention of re-thrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis.

In addition to its effects on the coagulation process, thrombin is known to activate a large number of cells (such as neutrophils, fibroblasts, endothelial cells and smooth muscle cells). Therefore, the compounds of WO 97/23499, and ximelagatran in particular, may also be useful for the therapeutic and/or prophylactic treatment of idiopathic and adult respiratory distress syndrome, pulmonary fibrosis following treatment with radiation or chemotherapy, septic shock, septicemia, inflammatory responses, which include, but are not limited to, edema, acute or chronic atherosclerosis such as coronary arterial disease, cerebral arterial disease, peripheral arterial disease, reperfusion damage, and restenosis after percutaneous trans-luminal angioplasty (PTA).

Compounds of WO 97/23499, and ximelagatran in particular, that lead to inhibition of trypsin and/or thrombin may also be useful in the treatment of pancreatitis.

According to a further aspect of the present invention, there is provided a method of treatment of a condition where inhibition of thrombin is required which method comprises administration of a therapeutically effective amount of a compound of WO 97/23499, and ximelagatran in particular, or a pharmaceutically acceptable salt thereof, to a person suffering from, or susceptible to such a condition, which person has been previously tested for an “ALAT susceptibility allele”.

The compounds of WO 97/23499, and ximelagatran in particular, will normally be administered orally, buccally, rectally, dermally, nasally, tracheally, bronchially, by any other parenteral route or via inhalation, in the form of pharmaceutical preparations comprising the prodrug either as a free base, or a pharmaceutical acceptable non-toxic organic or inorganic acid addition salt, in a pharmaceutically acceptable dosage form. Depending upon the disorder and patient to be treated and the route of administration, the compositions may be administered at varying doses.

The compounds of WO 97/23499, and ximelagatran in particular, may also be combined and/or co-administered with any antithrombotic agent with a different mechanism of action, such as the antiplatelet agents acetylsalicylic acid, ticlopidine, clopidogrel, thromboxane receptor and/or synthetase inhibitors, fibrinogen receptor antagonists, prostacyclin mimetics and phosphodiesterase inhibitors and ADP-receptor (P₂T) antagonists.

The compounds of WO 97/23499, and ximelagatran in particular, may further be combined and/or co-administered with thrombolytics such as tissue plasminogen activator (natural or recombinant), streptokinase, urokinase, prourokinase, anisolated streptokinase plasminogen activator complex (ASPAC), animal salivary gland plasminogen activators, and the like, in the treatment of thrombotic diseases, in particular myocardial infarction.

According to a further aspect of WO 97/23499 there are provided suitable pharmaceutical formulations. Suitable daily doses of the compounds of WO 97/23499, and ximelagatran in particular, (and especially ximelagatran in a form disclosed in WO 00/14110), in therapeutical treatment of humans are about 0.001-100 mg/kg body weight at peroral administration and 0.001-50mg/kg body weight at parenteral administration.

The compounds of WO 97/23499, and ximelagatran in particular, are inactive per se to thrombin, trypsin and other serine proteases. The compounds thus remain inactive in the gastrointestinal tract and the potential complications experienced by orally administered anticoagulants which are active per se, such as bleeding and indigestion resulting from inhibition of trypsin, may thus be avoided.

Furthermore, local bleeding associated with and after parenteral administration of an active thrombin inhibitor may be avoided by using the compounds of WO 97/23499, and ximelagatran in particular. Thus, according to a further aspect of the invention there is provided a method of treatment comprising:
(a) selecting a patient in need of anti-thrombotic treatment, the patient's genome having been identified as bearing an adenine at position 102 (according to SEQ ID NO: 1), or an allele of a polymorphism in linkage disequilibrium with D′>0.9 therewith, on at least one chromosomal copy; and
(b) treating the patient with a compound that inhibits or blocks thrombin.
   In alternate embodiments, the compound that inhibits or blocks thrombin is ximelagatran or melagatran.

According to a further aspect of the invention there is provided a method of treatment comprising:
(a) selecting a patient in need of anti-thrombotic treatment, the patient's genome having been identified as bearing, on at least one chromosomal copy, an adenine at position 102 (according to SEQ ID NO: 1), or a guanine at position 52 of SEQ ID NO:2, or a thymine at position 52 of SEQ ID NO:3, or an adenine at position 51 of SEQ ID NO:4; and
(b) treating the patient with ximelagatran

According to further aspects of the invention, there is provided a method of recommending a treatment, the method comprising:
(a) selecting a patient in need of anti-thrombotic treatment, the patient's genome having been identified as bearing an adenine at position 102 (according to SEQ ID NO: 1), or an allele of a polymorphism in linkage disequilibrium with D′>0.9 therewith, on at least one chromosomal copy; and
(b)treating the patient with a compound that directly or indirectly inhibits or blocks thrombin.

In particular embodiments, the compound that inhibits or blocks thrombin (directly or indirectly) is ximelagatran or melagatran.

According to a further aspect of the invention there is provided a method of treating a human in need of treatment with ximelagatran or tacrine comprising determining whether or not the human possesses an adenine at position 102 (according to SEQ ID NO: 1), or an allele of a polymorphism in linkage disequilibrium with D′>0.9 therewith, and if the human does possess an adenine at said position, or an allele of a polymorphism in linkage disequilibrium with D′>0.9 therewith, the human is administered ximelagatran or tacrine. In a preferred embodiment, both chromosomal copies comprise an adenine at the location according to position 102 of SEQ ID NO: 1, or the polymorphism in linkage disequilibrium with D′>0.9 therewith.

In an alternate embodiment, the patient is screened for the presence of a cytosine at position 102 (according to SEQ ID NO: 1) and the individual is treated with ximelagatran or tacrine if their genome lacks a cytosine at position 102 (according to SEQ ID NO: 1).

According to another aspect of the present invention there is provided a method of treating a human in need of treatment with the drug ximelagatran or tacrine, which method comprises:
i) determining the identity of SNPs rs2299257 in the human PON1 gene, or a polymorphism in linkage disequilibrium with D′>0.9 therewith,
ii) determining the status of the human by reference to the SNP present in (i); and,
ii) administering an effective amount of the drug.

In particular embodiments, the polymorphism in linkage disequilibrium with D′>0.9 to rs2299257 is selected from the group consisting of: rs1157745, rs662 and rs2269829. The status of the individual (i.e. likelihood of experiencing elevated ALAT following ximelagatran or tacrine administration) is assessed according to the particular nucleotide present at the SNP positions identified as taught herein.

According to another aspect of the present invention there is provided a pharmaceutical pack comprising the drug ximelagatran or tacrine and instructions for administration of the drug to humans diagnostically tested for a polymorphism in the PON1 gene, preferably at one or more of the 4 SNP positions specifically defined herein.

Antibodies can be prepared using any suitable method. For example, purified polypeptide may be utilized to prepare specific antibodies. The term “antibodies” is meant to include polyclonal antibodies, monoclonal antibodies, and the various types of antibody constructs such as for example F(ab′)₂, Fab and single chain Fv. Antibodies are defined to be specifically binding if they bind the allelic variant of PON1 (e.g. Q192R) with a Kₐ of greater than or equal to about 10⁷ M⁻¹. Affinity of binding can be determined using conventional techniques, for example those described by Scatchard et al., Ann. N.Y. Acad. Sci., (1949) 51:660.

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011; 4,902,614; 4,543,439 and 4,411,993; Monoclonal Antibodies, Hybridomas: A New Dimension in Biological Analyses, Plenum Press, Kennett, McKearn, and Bechtol (eds.), (1980).

Monoclonal antibodies for use in the invention can be produced using alternative techniques, such as those described by Alting-Mees et al., “Monoclonal Antibody Expression Libraries: A Rapid Alternative to Hybridomas”, Strategies in Molecular Biology (1990) 3:1-9, which is incorporated herein by reference. Similarly, binding partners can be constructed using recombinant DNA techniques to incorporate the variable regions of a gene that encodes a specific binding antibody. Such a technique is described in Larrick et al., Biotechnology, (1989) 7: 394.

Once isolated and purified, the antibodies may be used to detect the presence of antigen in a sample using established assay protocols, see for example “A Practical Guide to ELISA” by D. M. Kemeny, Pergamon Press, Oxford, England.

As noted above, a T at position 52 (according to SEQ ID NO: 3) encodes an arginine at amino acid position 192. Such variation in the polypeptide can either be detected enzymatically, or via use of a specific antibody, particularly a monoclonal antibody.

According to further aspects of the invention there is provided the use of ximelagatran or tacrine in the manufacture of a medicament for treating patients in need of anti-thrombotic or anticholinesterase treatment and whose genomes comprise comprises an adenine at position 102 (according to SEQ ID NO: 1), or an allele of a polymorphism in linkage disequilibrium with D′>0.9 therewith.

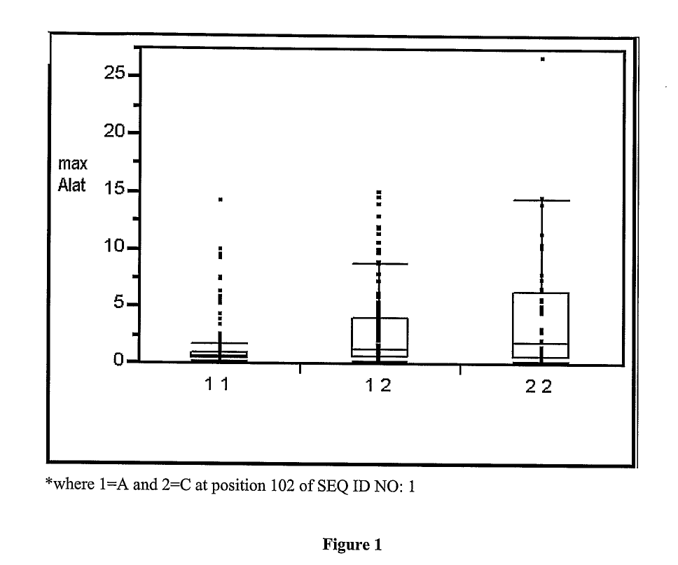

The invention will now be illustrated but not limited by reference to the following Examples and FIG. 1, which shows a box plot of rs2299257 genotype* (*where 1=A and 2=C at position 102 of SEQ ID NO: 1) versus maximum ALAT in cases and controls.

General molecular biology procedures can be followed from any of the methods described in “Molecular Cloning—A Laboratory Manual” Second Edition, Sambrook, Fritsch and Maniatis (Cold Spring Harbor Laboratory, 1989) or “Current Protocols in Molecular Biology Volumes 1-3, Edited by F M Asubel, R Brent, R E Kingston pub John Wiley 1998

### EXAMPLES

### Example 1

Subjects who had a transient increase of ALAT>3×ULN and thereafter returned to the baseline level at any time period during days 45-160 of treatment with ximelagatran (cases) were compared with subjects (controls) selected from the same studies but without ALAT increase during this period. In this analysis 74 cases and 169 controls were selected. Case-control status was used as the primary variable for statistical analysis. Max ALAT and AUC in the treatment interval 0-180 days were used for quantitative trait association analysis.

A single blood sample with informed consent was obtained from each of the subjects in the study. DNA was extracted from these samples using standard methodology and thousands of single nucleotide polymorphism (SNP) markers across the genome were genotyped.

The following standard methods were used for statistical analysis:
Differences in SNP genotype and allele frequencies between cases & controls
ANOVA of differences in max ALAT and AUC between SNP genotype groups
Logistic regression analysis of haplotype frequencies between cases & controls
Standard regression analysis of differences in max ALAT and AUC between haplotypes

The association results for each gene were summarised into a single statistic, p_min, which is simply the minimum p-value across all of the analyses for the gene. SNPs were ranked in terms of lowest p value.

The results of this analysis showed a highly significant association between a SNP in PON1 (rs2299257) and case-control status (p=1.14×10⁻⁵). The occurrence of a C at position 102 of SEQ ID NO: 1 was detected more frequently in cases than controls (see FIG. 1).

The same SNP was also significantly associated with maximum ALAT levels in a regression analysis (FIG. 1, p=1.19×10⁻⁵). Three other SNPs within PON1 (rs1157745, rs662 and rs2269829) were also highly significantly associated with case-control status (p=1.52×10^{-5,} p=1.52×10⁻⁵ and 4.26×10⁻⁵ respectively). Details of these associations are shown in Table 2.

TABLE 2
SNP ids, minimum P values, associated alleles and D′ between SNPs

| | | | | | Allele | | |
|---|---|---|---|---|---|---|---|
| SNP id | | | | | associated with | | D′ with |
| (rs number) | P_min | 5′ flank | SNP | 3′ flank | elevated ALAT | position | 2299257 |
| 2299257 | 1.14 × | AATGCA | A/C | AATTT | C | 102 of | — |
| | 10⁻⁵ | GAAG | | ATAGA | | SEQ01 | |
| 1157745 | 1.52 × | GAGAA | G/T | GTGA | T | 52 of | 1 |
| | 10⁻⁵ | GCATT | | TTGCT | | SEQ02 | |
| | | | | C | | | |
| 662 | 1.52 × | CTCCC | C/T | GTAA | C | 52 of | 1 |
| | 10⁻⁵ | AGGAT | | GTAG | | SEQ03 | |
| | | | | GG | | | |
| 2269829 | 4.26 × | GGCTT | A/G | AGAG | G | 51 of | 1 |
| | 10⁻⁵ | GGATC | | AAGT | | SEQ04 | |
| | | | | CT | | | |

### Example 2

Twenty one subjects treated with tacrine who had an increase of ALAT>3×ULN (cases) were compared with 43 subjects (controls) treated in the same way but without ALAT increase (total 64 individuals). Mean ALAT levels were used as the primary variable for statistical analysis.

A single blood sample with informed consent was obtained from each of the subjects in the study. DNA was extracted from these samples using standard methodology, and statistical analysis consisted of ANOVA of differences in mean ALAT between SNP genotype groups.

The results of this analysis showed a weak association between a SNP in PON1 (rs662) and mean ALAT levels. Mean ALAT levels were only 2.3×ULN in individuals with a TT genotype compared with 3.0×ULN in individuals with the CT genotype and 3.1 with the CC genotype. Hence, the occurrence of a C at position 102 of SEQ ID NO: 3 was detected more frequently in cases than controls (see Table. 3). Because of the low numbers of patients available for study, this association was only weakly statistically significant (p=0.08).

TABLE 3
Association of SNP rs662 with elevated ALAT in patients treated with tacrine

| | | | | | Allele | |
|---|---|---|---|---|---|---|
| SNP id | | | | | associated with | |
| (rs number) | P_min | 5′ flank | SNP | 3′ flank | elevated ALAT | position |
| 662 | 0.0807 | CTCCC | C/T | GTAAG | C | 52 of SEQ ID |
| | | AGGAT | | TAGGG | | NO: 3 |

Since the other SNPs in PON1 are correlated with rs 662 (r squared ≧0.59), determination of an individual's carrier status for the A allele at rs2299257, the G allele at rs1157745 or the A allele at rs2269829 can also be used to predict the likelihood that an individual can be treated with tacrine without experiencing elevated ALAT≧3×ULN.

In conclusion, these results suggest that determination of an individual's carrier status for the A allele at rs2299257 (position 102 of SEQ ID NO: 01) can be used to predict the likelihood that an individual can be treated with ximelagatran or tacrine without experiencing elevated ALAT≧3×ULN. Similarly, testing for G allele at rs 1157745 or T allele at rs662 or A allele at rs2269829 can be used to predict the likelihood that an individual can be treated with ximelagatran or tacrine without having a transient increase of ALAT≧3×ULN. Hence, a test that determined the carrier status of an individual for the particular nucleotide at these allelic positions could be used to determine the suitability of an individual for ximelagatran or tacrine treatment.

### Example 3

PON1 activity can be measured by a 2-step enzymatic assay of plasma. This is done by determining the rate at which the individual's plasma hydrolyses diazoxon and plotting this against the rate at which it hydrolyzes paraxon (Richter & Furlong, Pharmacogenetics, 9, 745 (1999). This method is able to predict L192R genotype (encoded by rs662) of an individual and also takes into account increased enzymatic activity caused by increased transcription and/or environmental factors.

1. A method of diagnosis comprising:
a) providing a biological sample from a human identified as being in need of treatment with a drug likely to interact with paraoxonase, wherein the sample comprises a nucleic acid encoding PON1 gene;
b) testing the nucleic acid for the presence, on at least one allele, of either
i) a nucleotide A at the position corresponding to position 102 of SEQ ID NO: 1, or
ii) an allele of a polymorphism in linkage disequilibrium with a D′>0.9 with (i); and
c) if either (i) or (ii) is found in at least one allele, diagnosing the human as being in the low likelihood category of having raised ALAT levels after treatment with the drug likely to interact with paraoxonase.

2. The method as claimed in claim 1, wherein the allele of a polymorphism in linkage disequilibrium with a D′>0.9 with (i) is selected from the group consisting of: G at position 52 of SEQ ID NO:2, T at position 52 of SEQ ID NO:3, and A at position 51 of SEQ ID NO:4.

3. The method as claimed in claims 1 or 2, wherein if in (c) (i) or (ii) is not found in at least one allele the human is diagnosed as being in the high likelihood category of having raised ALAT levels after treatment with a drug likely to interact with paraoxonase.

4. A method for sub-typing a human individual according to their likelihood status of experiencing elevated ALAT following administration of a drug likely to interact with paraoxonase, comprising the steps of:
a) treating nucleic acid from a sample that has been removed from the individual so as to identify the nucleotides present at one or more of the PON1 gene SNPs selected from the group consisting of rs2299257, rs1157745, rs662 and rs2269829; and
b) assigning the individual to a particular sub-type based on likelihood of experiencing elevated ALAT following administration of a drug likely to interact with paraoxonase, according to the nucleotide(s) detected in step a).

5. The method as claimed in claim 4, wherein the presence of adenine (A) nucleotide at rs2299257 or guanine (G) nucleotide at rs1157745 or thymine (T) nucleotide at rs662 or adenine (A) nucleotide at rs2269829, on at least one allele, puts that individual into a low likelihood sub-type of experiencing elevated ALAT following administration of a drug likely to interact with paraoxonase.

6. The method as claimed in claim 4, wherein the presence, on both alleles, of cytosine (C) nucleotide at rs2299257 or thymine (T) nucleotide at rs1157745 or cytosine (C) nucleotide at rs662 or guanine (G) nucleotide at rs2269829, puts that individual into a high likelihood sub-type of experiencing elevated ALAT following administration of a drug likely to interact with paraoxonase.

7. (canceled)

8. (canceled)

9. (canceled)

10. (canceled)

11. An in vitro diagnostic kit for screening for a genetic predisposition to elevated ALAT levels following administration of a drug likely to interact with paraoxonase, which kit comprises components for determining the identity of the nucleotide present at one or more of SNPs rs2299257, rs1157745, rs662 and rs2269829 in the human PON1 gene.

12. The kit as claimed in claim 11, wherein the kit components include allele-specific amplification primers or allele-specific hybridisation probes capable of determining the identity of the nucleotide bases at the SNP locations.

13. A method of treatment comprising:
a) selecting a patient in need of treatment with a drug likely to interact with paraoxonase, the patient's genome having been identified as bearing an adenine at position 102 (according to SEQ ID NO: 1), or an allele of a polymorphism in linkage disequilibrium with D′>0.9 therewith, on at least one chromosomal copy; and
b) treating the patient with an appropriate compound.

14. The method as claimed in claim 13, wherein in step (b) the patient is treated with ximelagatran or tacrine.

15. A method of treating a human in need of treatment with a drug likely to interact with paraoxonase, which method comprises:
a) determining the identity of SNPs rs2299257 in the human PON1 gene, or a polymorphism in linkage disequilibrium with D′>0.9 therewith,
b) determining the status of the human by reference to the SNP present in (a); and,
c) administering an effective amount of the drug.

16. The method as claimed in claim 15, wherein the polymorphism in linkage disequilibrium with rs2299257 is selected from: rs1157745, rs662 and rs2269829.

17. (canceled)

18. (canceled)

19. The method as claimed in claim 1, 4, 13 or 15 wherein the drug likely to interact with paraoxonase is selected from ximelagatran or tacrine.

20. The kit as claimed in claim 11, wherein the drug likely to interact with paraoxonase is selected from ximelagatran or tacrine
